(19) United States
(12) Patent Application Publication
(10) Pub. No.: US 2017/0239333 A1
(43) Pub. Date: Aug. 24, 2017

(54) METHODS FOR PREVENTING PREMATURE FOLLICLE ACTIVATION

(71) Applicant: TEL HASHOMER MEDICAL RESEARCH INFRASTRUCTURE AND SERVICES LTD., Ramat Gan (IL)

(72) Inventors: Dror MEIROW, Jerusalem (IL); Hadassa RONESS, Neve Daniel (IL)

(73) Assignee: TEL HASHOMER MEDICAL RESEARCH INFRASTRUCTURE AND SERVICES LTD., Ramat Gan (IL)

(21) Appl. No.: 15/506,496

(22) PCT Filed: Aug. 30, 2015
(86) PCT No.: PCT/IL2015/050872

§ 371 Date: Feb. 24, 2017

Related U.S. Application Data
(60) Provisional application No. 62/044,259, filed on Aug. 31, 2014.

(51) Int. Cl. A61K 38/22

(52) U.S. Cl. CPC A61K 38/22

(57) ABSTRACT

Provided are compositions and methods for preventing premature follicle activation and loss induced by acute insults, such as, medical treatment, a disease or a disorder, thereby preserving fertility in a subject.

### FIELD OF THE INVENTION

The present invention relates to compositions and methods for preventing premature follicle activation and loss induced by acute insults, such as, medical treatment, a disease or a disorder, thereby preserving fertility in a subject.

### BACKGROUND OF THE INVENTION

The ovarian primordial follicle pool in humans is established during embryonic development. This pool constitutes the complete supply of oocytes that have the potential to ovulate through life. The population of primordial (non-growing) follicles (‘reserve’) containing diplotene oocytes is arrested in the first meiotic prophase. A ‘reserve’ of primordial follicles is the number of primordial follicles at any given age and is ultimately depleted by continuous recruitment and degeneration until exhausted. After primordial follicle development is initiated, a small number of the follicles are destined to ovulate while the rest undergo atresia. The factors that control the initiation of primordial follicle development are crucial for female fertility.

Follicle reservoir destruction is a major side effect of numerous acute insults, including chemotherapy treatments in young female cancer patients (Meirow et al., Human Reprod., 22(6):1626-33, 2007). The chemotherapy treatment may cause premature menopause and infertility. Alkylating agents such as cyclophosphamide (Cy) cause a loss of the population of non-growing follicles (NGF), resulting in a reduction in ovarian reserve. It was shown by some of the inventors of the present invention that the loss of ovarian reserve is due to accelerated primordial follicle activation (Kalich-Philosoph et al. Sci Transl Med, 5(185):185, 2013; Roness et al., Cell Cycle, 12(20): 3245-3246, 2013).

Current options for female fertility preservation (including embryo, oocyte and ovarian tissue cryopreservation) are limited by patient age, status or available timeframe before treatment and necessitate invasive procedures. Pharmacological agents that can prevent the loss of follicles at the time of treatment would provide significant advantages over existing fertility preservation techniques in that they would be suitable for patients of all ages and life stages, would not require invasive surgical procedures or subsequent use of assisted reproductive technologies, and would prevent the myriad endocrine related side effects of premature ovarian failure (POF) other than infertility.

Roness et al. (Hum Reprod Update., 20(5):759-774, September-October 2014) outline the impact and mechanisms of cytotoxic drugs on the various cell-types of the ovary, and review the recent developments in the field of fertility preservation for female cancer patients.

Anti-mullerian hormone (AMH) is produced by the granulosa cells of early growing follicles. AMH serum levels are currently used as a marker of ovarian follicle reservoir. Studies have shown that AMH participates in selection points of follicle development (Skinner, Hum Reprod Update., 11(5):461-471, 2005). AMH was also shown to have a role in maintaining primordial follicle dormancy under physiological conditions (Reddy et al., Trends in Endoc. & Metabol., 21(2):96-103, 2010). However, studies conducted in vitro (2-day-old mouse ovarian culture) and in AMH-knockout female mice did not examine the effect of exogenous AMH (Durlinger et al. Reproduction (2002) 124, 601-609), while other studies concluded that exogenous AMH does not affect the number of primordial follicles (Durlinger et al., Endocrinology, 143(3):1076-1084, 2002).

There remains an unmet need for therapeutic approaches using pharmacological agents rather than invasive procedures, for preserving the oocyte pool and preventing undesirable and premature follicle activation and loss induced by external acute insults, including, treatments, medical procedures, diseases or disorders.

### SUMMARY OF THE INVENTION

The present invention provides pharmaceutical compositions, kits and methods for protecting fertility under or following acute insults using AMH, including AMH agonist or antiMIR of AMH or a combination thereof. Thus, the methods of the invention are useful for preventing premature follicle activation and loss, inhibiting undesired or premature activation of follicles, preserving the depot of primordial follicles, postponing premature menopause, reducing the side effects associated with premature menopause, treating diseases and disorders associated with premature follicle activation and/or loss and preserving the population of non-growing follicles during treatment that induce follicle loss through activation.

The term “premature follicle activation” is interchangeable with the term “artificially induced follicle activation” and “induced follicle activation” and refers to accelerated and/or premature follicle activation and follicle loss (also termed ‘follicle burn-out’) which is induced by an acute damage, such as medical treatment, including, but not limited to treatment with chemotherapeutic agents, radiotherapy, ovary transplantation among other medical treatments that may induce follicle burn out. These terms also encompass early, premature, follicle activation and loss induced by diseases or disorders.

Unexpectedly, as exemplified herein below, AMH induced protection of primordial follicles in ovaries treated with a chemotherapeutic drug known to reduce follicle count. Moreover, ovaries exposed to chemotherapy and AMH had an improved ratio of growing to dormant follicles, compared to ovaries exposed to chemotherapy alone.

Accordingly, the pharmaceutical compositions, kits and methods of the invention provide a therapeutic platform for reducing complications related to medical treatments and diseases, which cause premature menopause. In addition, the pharmaceutical compositions, kits and methods of the invention are effective in providing therapy to preserve the fertility of women suffering from a disease or disorder that accelerates follicle activation.

There is provided, in some embodiments, a method of inhibiting premature follicle activation comprising administering to a subject in need thereof a therapeutically effective amount of a pharmaceutical composition comprising a compound selected from the group consisting of anti-mullerian hormone, anti-mullerian hormone agonist, and antiMIR of anti-mullerian hormone, wherein the follicle activation is induced by an acute insult.

In some embodiments, the acute insults comprises at least one of treatment, agent, disease or a combination thereof.

In some embodiments, the compound is anti-mullerian hormone (AMH).

In some embodiments, the treatment is transplantation of ovarian tissue or whole ovary. In some embodiments, said agent induces follicle loss. In some embodiments, said agent is an anti-cancer agent. In some embodiments, said agent is a chemotherapeutic drug. In some embodiments, said treatment is radiotherapy. In some embodiments, said acute insult comprises chemotherapy, radiotherapy and a combination thereof. Each possibility is a separate embodiment of the invention.

In some embodiments, said administering said pharmaceutical composition is carried out in combination with said acute insult. In some embodiments, said administering said pharmaceutical composition is carried out prior to said acute insult.

In some embodiments, the disease is a benign disease or disorder associated with chemotherapy treatments, such as, lupus, rheumatoid arthritis and skin diseases. In some embodiments, the disease is selected from the group consisting of: endometriosis, galactosemia, Turner syndrome and an autoimmune disease. In some embodiments, the autoimmune disease is selected from lupus and multiple sclerosis.

In some embodiments, the disease is an accelerated follicle activation disorder. In some embodiments, the accelerated follicle activation disorder is endometriosis.

In some embodiments, the subject in need thereof is a subject at perimenopause.

In some embodiments, the method of inhibiting premature follicle activation further comprises transplanting ovarian tissue or whole ovary, wherein the acute insult is transplantation of ovarian tissue or whole ovary. In some embodiments, the graft (ovarian tissue or whole ovary) is coated prior to transplantation. In some embodiments, said pharmaceutical composition is administered prior to said transplanting. In some embodiments, said pharmaceutical composition is administered during the transplantation. In some embodiments, said pharmaceutical composition is administered during the transplantation, wherein said graft is covered with alginate.

In some embodiments, the method further comprises administering to said subject at least one follicle reserve protective compound.

There is provided, in some embodiments, use of a pharmaceutical composition comprising anti-mullerian hormone, anti-mullerian hormone agonist or antiMIR of anti-mullerian hormone for inhibiting premature follicle activation induced by an acute insult.

There is provided, in some embodiments, use of a pharmaceutical composition comprising anti-mullerian hormone, anti-mullerian hormone agonist or antiMIR of anti-mullerian hormone for inhibiting artificially induced follicle activation.

The terms “induced”, “artificially induced” and “acute insult” as used herein are interchangeable and refer to follicle activation and loss of ovarian reserve which are induced by external cause(s) or diseases and not as a result of normal physiological conditions. Disease that may be classified as acute insults include an accelerated follicle activation disease or disorder, such as, endometriosis, galactosemia and Turner syndrome. Other diseases that may be classified as acute insults include autoimmune diseases such as, lupus and multiple sclerosis, which are treated using chemotherapy agents known to interfere with fertility.

There is provided, in some embodiments, use of anti-mullerian hormone, anti-mullerian hormone agonist, antiMIR of anti-mullerian hormone, or pharmaceutical composition comprising same, for the manufacture of a medicament for inhibiting, attenuating or preventing artificially induced follicle activation and follicle loss (burn out).

There is provided, in some embodiments, a kit for inhibiting induced follicle activation in a subject in need thereof comprising:
(i) a first packaging containing a pharmaceutical composition comprising a compound selected from the group consisting of anti-mullerian hormone, anti-mullerian hormone agonist, and antiMIR of anti-mullerian hormone; and
(ii) written instructions of use of said pharmaceutical composition for inhibiting follicle activation induced in said subject.

In some embodiments, the kit further comprises a second packaging containing a pharmaceutical composition comprising at least one agent that induces follicle loss.

In some embodiments, the kit further comprises a third packaging containing at least one follicle reserve protective compound.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. It is intended that the embodiments and figures disclosed herein are to be considered illustrative rather than restrictive. The figures are listed below.

FIG. 1 shows count of primordial (white columns) and growing (black columns) follicles in whole ovaries cultured with medium alone (control; No Rx), medium and 200 ng/ml AMH (AMH), in the presence of phosphoramide mustard (PM) for 4 hours followed by medium alone, and initially cultured with PM together with 200 ng/ml AMH (PM+AMH). **p<0.01 No Rx compared with PM, ***p<0.001 PM+AMH compared with PM alone.

FIG. 2 shows the ratio of growing to dormant (primordial stage) follicles in each of the treatment groups presented in FIG. 1. *p<0.05 No Rx compared with PM, and PM+AMH compared with PM alone.

### DETAILED DESCRIPTION OF THE INVENTION

Studies of fetal, neonatal, and adult human ovaries have shown that several millions of non-growing follicles (NGF) are established at around five months of gestational age. This number declines to the point where approximately 1,000 remain at the onset of menopause, which occurs at an average age of 50-51 years. It was further estimated that for 95% of women by the age of 30 years only 12% of their maximum pre-birth NGF population remains and by the age of 40 years only 3% remains. Although about one million oocytes are present at birth in the human ovary, only about 500 (about 0.05%) of these ovulate, where the rest are wasted.

Destruction of ovarian follicle reserve is a major side effect of various acute insults, including, but not limited to, chemotherapy. The impact of chemotherapy on fertility is directly dependent on the survival or loss of the dormant oocytes in the primordial follicles that comprise the ovarian follicle reserve. Chemotherapy induces distinct short and long-term effects on the ovary. The immediate effect, occurring during treatment, includes temporary amenorrhea. The greater long-term effect includes damage caused to the primordial follicle pool. Though total loss of the primordial follicle population may occur, resulting in immediate and permanent sterilization, the more common damage is partial loss of the primordial follicle reserve. If sufficient primordial follicles remain, the amenorrhea induced by the loss of the growing follicle population may be short lived. However, the reduction of the primordial follicle pool decreases the remaining window of fertility available to the patient, resulting in permanent amenorrhea and premature menopause.

Most classes of cytotoxic drugs target rapidly dividing cells, interrupting essential cell processes and arresting cellular proliferation. Alkylating agents are not cell-cycle specific and are cytotoxic even when cells are at rest although proliferating cells are known to be more sensitive to their effects. Histological studies on human tissue show that chemotherapy causes a drastic loss of primordial follicle stockpiles. Paclitaxel and cisplatin have been observed to decrease the number of primordial follicles in mice and rats which may be due to a direct effect of the treatment on follicles or an indirect effect via another cell type such as stroma.

Thus, the present invention provides pharmaceutical compositions, kits and use thereof for inhibiting or preventing follicle activation induced by acute insults, including, treatment(s), agents, disease(s) and/or disorder(s). The compositions and kits of the invention comprise AMH, including AMH agonist or antiMIR of AMH and a combination thereof. The compositions and kits of the invention may be used prior to or in combination with anti-cancer therapy. The compositions of the invention may be used in any disorder or therapeutically procedure that involves premature or undesired follicle activation and protect the PI3K pathway by inhibiting, attenuating or preventing its activation.

The terms “follicle activation”, “initiation of follicle growth” and “initial recruitment of follicle” as used herein are interchangeable and generally refer to the transition of dormant/primordial follicles into growing follicles.

The terms “premature follicle activation”, “early follicle activation” and “follicle burn out” a used herein are interchangeable and refer to processes induced by acute insults which may eventually cause, or result in, loss of fertility. The aforementioned processes may results with menopause earlier than expected.

Anti-mullerian hormone, also termed hereinafter “AMH”, typically refers to a protein designated by NCBI Accession No.: P03971. It has been also termed Müllerian inhibiting factor (MIF), Müllerian-inhibiting hormone (MIH), and Müllerian-inhibiting substance. The present invention encompasses the full AMH sequence, homologs, analogs, variants and derivative of the AMH protein or a fragment thereof, with the stipulation that the AMH activity is preserved. A mathematical model simulating the female reproductive cycle, predicted that AMH could be used to delay, naturally, menopause (Margolskee et at, J. Theor. Biol., 326:21-35, February 2013).

Without being bound by any theory or mechanism, AMH inhibits or prevents follicle activation by inhibiting or preventing recruitment of primordial follicles into the pool of growing follicles, thereby preventing undesired acceleration effect on growing follicle resulting in follicle exhaustion, as for example induced by a disease, a syndrome, invasive procedures and/or medicaments, such as, chemotherapy. Under normal physiological conditions, AMH protects the reserve of primordial follicles. Under acute insult the follicle reserve may undergo apoptosis and/or may be reduced for lack of nutrients due to destructions of the vascular system that nourishes the follicles. Unexpectedly, as shown herein, AMH overcomes, inhibits, prevents and/or bypasses the destructive effect of the acute insult. The effect of exogenous AMH, as disclosed herein, is pronounced even in parallel to the acute insult, and before or after the acute insult. Thus, in the event that the acute insult is a disease, AMH or its derivatives, may be used in parallel to the disease. In the event that the acute insult is a medical procedure or treatment, treatment with AMH or its derivatives may be carried out before, after or in combination with the medical procedure or treatment.

In some embodiments, the present invention provides a method of inhibiting induced follicle activation comprising administering to a subject in need thereof a therapeutically effective amount of a pharmaceutical composition comprising a compound selected from the group consisting of anti-mullerian hormone, anti-mullerian hormone agonist, and antiMIR of anti-mullerian hormone.

The term “agonist” as used herein refers to any chemical substance, a fragment of AMH protein, a derivative of AMH or a modified AMH protein, which capable of activating the AMH receptor, resulting with the inhibition of follicle activation induced by an acute insult.

As used herein and further detailed below, the term “inhibiting follicle activation” or “preventing follicle activation” refers to a transient or permanent condition wherein some or all follicles are maintained in their primordial stage.

The term “antiMIR” refers to contiguous nucleic acids, DNA or RNA, which are complementary to micro-RNA or miRNA. The antiMIR binds to the miRNA and inhibits the silencing/degrading activity it has upon the mRNA of a target gene. This results in elevation of the target gene expression. The antiMIR of the invention is targeted for miRNA that silence the AMH gene. In some embodiments, the present invention provides a method of preventing artificially induced follicle activation, comprising the step of administering antiMIR of AMH.

The term “antiMIR of AMH” refers to a molecule that inhibits AMH silencing by miRNA.

The term “complementary” in the context of the present invention refers to antiMIR sequence that has at least 90%, 95%, or 100% identity to a complementary sequence of miRNA of AMH.

The term “inhibiting” as used herein includes, but is not limited to, preventing, attenuating, impeding, reducing to a certain extent, complete inhibition and/or partial inhibition.

As used herein, the term “therapeutically effective amount” refers to an amount of a formulation or composition which is effective to inhibit or prevent, at least partially, follicle activation in a living organism to whom it is administered over some period of time.

The present invention further provides a method for inhibiting or preventing the induced follicle activation in a subject in need thereof by increasing the activity of the AMH receptor. Increasing the activity of an AMH receptor may be obtained, for example, by elevating the AMH or AMH agonist amounts. Administering AMH per se is one approach for elevating AMH amount. Another approach is by inducing overexpression of a gene encoding for AMH. Overexpression of AMH could be achieved by gene therapy mediated by adenovirus and lentivirus vectors.

The AMH protein hormone may be isolated and purified by methods selected based on properties revealed by its sequence. Purification can be achieved by protein purification procedures such as chromatography methods (gel-filtration, ion-exchange and immunoaffinity), by high-performance liquid chromatography (HPLC, RP-HPLC, ion-exchange HPLC, size-exclusion HPLC, high-performance chromatofocusing and hydrophobic interaction chromatography) or by precipitation (immunoprecipitation). Polyacrylamide gel electrophoresis can also be used to isolate the AMH protein based on the molecular weight of the protein, charge properties and hydrophobicity. For example, Picard et al. describes an improved method for the purification of anti-Müllerian hormone from incubation medium of bovine fetal testes (Mol Cell Endocrinol., 1984, 34(1):23-29).

According to alternative embodiments, AMH or its equivalents may be produced by the use of recombinant DNA techniques as are well known to one skilled in the art. Nucleic acid sequences which encode for the proteins of the invention may be incorporated in a known manner into appropriate expression vectors (i.e. recombinant expression vectors). Possible expression vectors include, but are not limited to, cosmids, plasmids, or modified viruses (e.g. replication defective retroviruses, adenoviruses and adeno-associated viruses, lentiviruses, herpes viruses, poxviruses), so long as the vector is compatible with the host cell used. The expression “vector . . . compatible with the host cell” is defined as contemplating that the expression vector(s) contain a nucleic acid molecule of the invention and attendant regulatory sequence(s) selected on the basis of the host cell(s) to be used for expression, said regulatory sequence(s) being operatively linked to the nucleic acid molecule. “Operatively linked” is intended to mean that the nucleic acid is linked to regulatory sequence(s) in a manner which allows expression of the nucleic acid. Suitable regulatory sequences may be derived from a variety of sources, including bacteria, fungal, or viral genes (for example, see the regulatory sequences described in Goeddel, Gene Expression Technology: Methods in Enzymology 185, Academic Press, San Diego, Calif. (1990)). Selection of appropriate regulatory sequence(s) is dependent on the host cell(s) chosen, and may be readily accomplished by one of ordinary skill in the art. Examples of such regulatory sequences include the following: a transcriptional promoter and enhancer, RNA polymerase binding sequence, or a ribosomal binding sequence (including a translation initiation signal). Depending on the host cell chosen and the expression vector employed, other additional sequences (such as an origin of replication, additional DNA restriction sites, enhancers, and sequences conferring inducibility of transcription) may be incorporated into the expression vector.

It is to be understood that the pharmaceutical compositions, kits and methods of the invention are directed for treating women. Thus, the terms ‘subject’ and ‘subject in need thereof’ refer to females in their reproductive, fertile, years, including, women and adolescent children. The term ‘reproductive years’ refers in general to the age following puberty and prior to menopause.

In some embodiment, the subject is a subject having a disease or disorder associated with loss of follicle activation and/or loss or reduced fertility.

In some embodiment, the subject is a subject having a disease or disorder associated with loss of follicle activation and/or loss or reduced fertility which are induced by a medical treatment.

In some embodiments, the subject is undergoing treatment with an agent that induces follicle loss. In some embodiments, said agent is a chemotherapeutic agent.

In some embodiments, the subject is a subject having cancer. In some embodiments, the subject having cancer is being treated with anti-cancer therapy prior to and/or in parallel to treatment for inhibiting follicle activation. In some embodiments, the anti-cancer therapy is chemotherapy.

In other embodiments, the anti-cancer therapy is radiotherapy. In some embodiments, the anti-cancer treatment results in accelerated or premature follicle activation.

The term “cancer” is used herein in its broadest sense and refers to a family of diseases characterized by uncontrolled cell growth. It includes, but is not limited to, adrenocortical carcinoma, anal cancer, bladder cancer, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors, extracranial germ cell tumor, eye cancer, intraocular melanoma, gallbladder cancer, gastric cancer, germ cell tumor, extragonadal germ cell tumor, gestational trophoblastic tumor, head and neck cancer, hypopharyngeal cancer, islet cell carcinoma, laryngeal cancer, acute lymphoblastic leukemia, oral cavity cancer, liver cancer, lung cancer, small cell lymphoma, AIDS-related lymphoma, central nervous system (primary) lymphoma, cutaneous T-cell lymphoma, hodgkin's disease, non-hodgkin's disease, malignant mesothelioma, melanoma, merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine pancreatic cancer, paranasal sinus and nasal cavity cancer, parathyroid cancer, pheochromocytoma cancer, pituitary cancer, plasma cell neoplasm, rhabdomyosarcoma, rectal cancer, renal cell cancer, salivary gland cancer, sezary syndrome, kaposi's sarcoma, skin cancer, small intestine cancer, soft tissue sarcoma, thymoma, malignant, thyroid cancer, urethral cancer, uterine cancer, sarcoma, unusual cancer of childhood, vaginal cancer, vulvar cancer, or wilms' tumor.

In some embodiments, the subject in need is having an accelerated follicle activation disease or disorder. In some embodiments, the disease is a genetic disorder such as Turner syndrome. In other embodiments, the disease is Galactosemia. In other embodiments, the disease is endometriosis.

Turner syndrome refers to a chromosomal condition that affects development in females. Turner syndrome occurs when one normal X chromosome is present in a female's cells and the other sex chromosome is missing or structurally altered. Turner syndrome is characterized by an early loss of ovarian function and accelerated follicle activation may be one of the causes for this phenomenon.

Galactosemia is an inherited disorder characterized by inability to metabolize the sugar galactose properly. One of the symptoms of Galactosemia is accelerated follicle activation.

In some embodiments, the present invention provides a method of inhibiting follicle activation in subject undergoing ovary transplantation, the method comprises administering to a subject a therapeutically effective amount of a pharmaceutical composition comprising a compound selected from the group consisting of anti-mullerian hormone, anti-mullerian hormone agonist, and antiMIR of anti-mullerian hormone, following, prior to, or in combination with ovarian tissue or whole ovary transplantation.

The term ‘ovary transplantation’ as used herein refers to transplantation of the whole ovary or of parts of the ovary, also termed herein ‘ovarian tissue’.

Transplantation of frozen thawed or fresh ovarian tissue or whole ovary is a delicate procedure aimed to restore fertility to patients who have lost ovarian follicle reserve or have poor quality follicles by delivering a stock of resting non growing follicles that can serve in the future to restore and maintain follicular activity and ovulations that may enable future reproduction. However, high portion of follicles delivered back to the body by transplantation disappear rapidly due to premature follicle activation. The present invention provides pharmaceutical compositions, kits and methods directed to increase graft survival, enable future pregnancy and prolong hormone secretion. Thus, in some embodiments, the pharmaceutical compositions, kits and methods of the invention are directed to subjects undergoing ovarian tissue transplantation or whole ovary transplantation.

The terms “non-growing follicles”, “NGF”, “resting follicles” and “dormant follicles” as used herein are interchangeable and refer to a depot of follicles prior to activation, which have the potential to become activated under suitable natural or artificial factors and conditions.

In some embodiments, said pharmaceutical composition is introduced to the patient together with the transplanted tissue/ovary. In some embodiments, said pharmaceutical composition is delivered topically, directly to the ovary. In some embodiments, said graft (ovarian tissue or whole ovary) is covered with alginate, encapsulated within alginate, or otherwise coated with alginate.

In some embodiments, the present invention provides a pharmaceutical composition comprising a compound selected from the group consisting of anti-mullerian hormone, anti-mullerian hormone agonist, and antiMIR of anti-mullerian hormone, for use in inhibiting follicle activation in subject in need thereof.

In some embodiments, the pharmaceutical composition further comprises a pharmaceutically acceptable carrier, excipient or diluent.

As used herein, a “pharmaceutical composition” refers to a preparation of one or more of the active ingredients described herein, for example, AMH molecule, AMH agonist, antiMIR of AMH, with non-active (inert) components, such as, physiologically suitable carriers and excipients. The purpose of a pharmaceutical composition is to facilitate administration of a compound to a subject.

Herein, the phrases “therapeutically acceptable carrier”, “physiologically suitable carriers and excipients” and “pharmaceutically acceptable carrier”, which may be used interchangeably, and refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

As used herein, a “carrier” refers to any substance suitable as a vehicle for delivering an amino acid or a nucleic acid molecule of the present invention to a suitable in vivo or in vitro site. As such, carriers can act as a pharmaceutically acceptable excipient of a therapeutic composition containing a molecule of the present invention. Carriers of the present invention include: (1) excipients or formularies that transport, but do not specifically target a nucleic acid molecule to a cell (referred to herein as non-targeting carriers); and (2) excipients or formularies that deliver an amino acid or nucleic acid molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Therapeutic compositions of the present invention can be sterilized by conventional methods.

The pharmaceutical compositions of the present invention may be manufactured by processes well known in the art for the preparation of pharmaceutically acceptable compositions intended for administration to a subject, e.g. by means of conventional mixing, dissolving, granulating, grinding, pulverizing, dragee-making, levigating, emulsifying, encapsulating, entrapping or lyophilizing processes.

The compositions described herein may be prepared such that an effective quantity of the active substance (e.g. AMH) is combined in a mixture with a suitable pharmaceutically acceptable vehicle as known in the art. On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids.

Furthermore, the pharmaceutical compositions according to the invention may comprise one or more stabilizers, such as, for example, carbohydrates including sorbitol, mannitol, starch, sucrose, dextrin and glucose, proteins such as albumin or casein, and buffers like alkaline phosphates.

In some embodiments, administering the pharmaceutical composition comprises administering via a route selected from the group consisting of: subcutaneous, topical, transdermal, oral, buccal, sublingual, sublabial, intradermal, intravaginal or combinations thereof. Each possibility is a separate embodiment of the invention.

In some embodiments, administering the pharmaceutical composition comprises direct delivery to the ovary. In some embodiments, administering the pharmaceutical composition comprises direct injection to the ovary. In some embodiments, administering the pharmaceutical composition comprises systemic administration.

In some embodiments, the pharmaceutical composition is administered by direct delivery to the ovary. In some embodiments, the pharmaceutical composition is delivered to each ovary. In some embodiments, the pharmaceutical composition is delivered to each ovary prior to initiation of the acute insult.

Administration of an “effective amount” of the pharmaceutical compositions of the present invention refers to administration of an amount effective at dosages and for periods of time, necessary to elicit a desired therapeutic response in a human. A therapeutically effective amount of a substance may vary according to the follicle activator factor or cause, age, sex, and weight of the recipient. Dosage regimes may be adjusted to provide the optimum therapeutic response. For example, several divided doses may be administered daily or on at periodic intervals, and/or the dose may be proportionally reduced as indicated by the exigencies of the therapeutic situation. The amount pharmaceutical composition for administration will depend on the route of administration, time of administration and varied in accordance with individual subject responses.

There is provided, in some embodiments, a kit for the inhibiting or preventing follicle activation in a subject in need thereof, the kit comprising:
(i) a first packaging containing a pharmaceutical composition comprising a compound selected from the group consisting of anti-mullerian hormone, anti-mullerian hormone agonist, and antiMIR of anti-mullerian hormone; and
(ii) written instructions for of use of said pharmaceutical composition for inhibiting follicle activation in said subject.

In some embodiments, the kit further comprises a second packaging containing a pharmaceutical composition comprising at least one anti-cancer agent.

In some embodiments, the kit further comprises a third packaging containing a pharmaceutical composition comprising at least one follicle reserve protective compound and a pharmaceutically acceptable carrier, diluent or excipient.

In some embodiments, the at least one follicle reserve protective compound comprises sphingosine-1-phosphate, Tamoxifene, GnRH, trichloro(dioxoethylene-O,O′) or a combination thereof.

In some embodiments, the pharmaceutical composition in the first packaging may further comprise at least one anticancer agent.

The term “kit” as used herein is interchangeable with the term package, and refers to packages of pharmaceutical formulations containing any one or more of anti-mullerian hormone, anti-mullerian hormone agonist, antiMIR of anti-mullerian hormone and further containing, together, or in a different packaging, the anticancer agent. Accordingly, the kit may be organized to indicate a single formulation or combination of formulations to be taken at each desired treatment regimen as specified in written instructions encompassed in the kit.

The kit may optionally contain instructions for administering the pharmaceutical composition to a subject having a disease associated with premature follicle activation or having a condition requiring to inhibit or attenuate follicle activation in order to protect fertility.

In some embodiments, the kit contains packaging or a container with each of said first and second and third pharmaceutical compositions, formulated for the desired delivery route. Suitably, the kit contains instructions on dosing and an insert regarding the active agent. Optionally, the kit may further contain instructions for monitoring circulating levels of product(s) and material(s) that may be used for evaluating treatment efficacy. For performing such evaluation assays that kit may further include reagents, well plates, containers, markers or labels, and the like. Such kits are readily packaged in a manner suitable for treatment of a desired indication. The kit may also contain instructions for use of a delivery device. Other suitable components to include in such kits will be readily apparent to one of skill in the art, taking into consideration the desired indication and the delivery route.

The compositions described herein can be a single dose or for continuous or periodic discontinuous administration. For continuous administration, the package or kit may include each of the pharmaceutical compositions in their dosage unit (e.g., solution, lotion, tablet, pill, or other unit described above or utilized in drug delivery), and optionally instructions for administering the doses daily, weekly, or monthly, for a predetermined length of time or as prescribed. When the pharmaceutical compositions are to be delivered periodically in a discontinuous fashion, the package or kit may include placebos during periods when the pharmaceutical compositions are not delivered. When varying concentrations of a composition, of the components of the composition, or the relative ratios of the components of the pharmaceutical composition or the ratio of the first pharmaceutical composition to the second pharmaceutical composition over time is desired, the package or kit may contain a sequence of dosage units which provide the desired variability.

A number of packages or kits are known in the art for dispensing pharmaceutical agents for periodic oral use. In some embodiments, the package has indicators for each period. In other embodiments, the package is a labeled blister package, dial dispenser package, or bottle.

The packaging means of a kit may itself be geared for administration, such as an inhaler, syringe, pipette, eye dropper, or other such apparatus, from which the pharmaceutical composition(s) may be applied to an affected area of the body, such as the arms, injected into a subject, or even applied to and mixed with the other components of the kit.

The compositions of the kit of the invention also may be provided in dried or lyophilized forms. When reagents or components are provided as a dried form, reconstitution generally is by the addition of a suitable solvent. It is envisioned that the solvent also may be provided in another packaging of the kit.

The kit of the present invention also will typically include a means for containing the vials in close confinement for commercial sale such as, e.g., injection or blow-molded plastic containers into which the desired vials are retained. Irrespective of the number or type of packages and as discussed above, the kit also may include, or be packaged with a separate instrument for assisting with the injection/administration or placement of the composition within the body. Such an instrument may be an inhaler, syringe, pipette, forceps, measuring spoon, eye dropper or any such medically approved delivery means.

In some embodiments, the pharmaceutical compositions of the kit are provided in the presence or absence of one or more of the carriers or excipients described above.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Examples

### Example 1: AMH Prevents Follicle Loss in Ovaries Treated with Chemotherapy

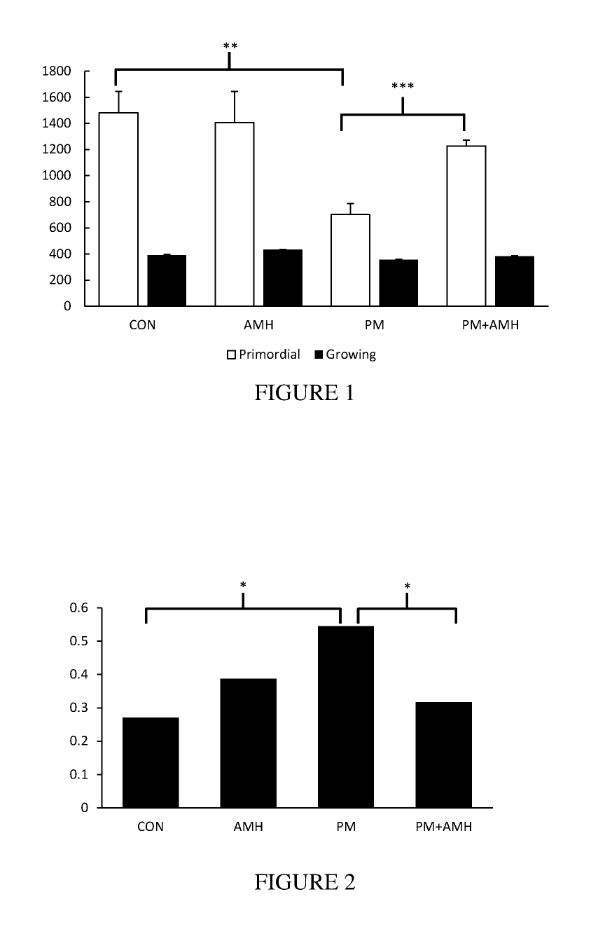

In mice, normal ovarian follicle dynamics are only fully established by approximately day 10 after birth and therefore ovaries from 12-day-old neonatal mice were used to examine the effect of AMH. The ovaries were cultured in-vitro in the presence of the cyclophosphamide metabolite, phosphoramide mustard (PM) for 4 hours, either with or without 200 ng/ml AMH (PM+AMH or PM, respectively) and then washed and continued culture was also with or without AMH. Control ovaries were cultured with medium alone (No Rx) or with only AMH (AMH). Ovaries were removed after day 4 and day 7 in culture and processed for histological analysis. The number of primordial and growing follicles was counted. No difference in primordial or growing follicle numbers was observed between ovaries cultured in regular media alone and ovaries cultured with media and AMH (FIG. 1). A significantly reduced numbers of primordial follicles was observed in ovaries exposed to PM alone compared with untreated ovaries (FIG. 1, white columns PM vs. Control; p<0.01). However, in ovaries exposed to PM together with AMH a significantly greater numbers of primordial follicles was observed relative to PM alone (FIG. 1, white columns PM+AMH vs. PM; p<0.001).

Ovaries from 12-day-old neonatal mice were further incubated with a different chemotherapy agent; cisplatin, with or without AMH. Control ovaries were cultured with medium alone or with AMH. Ovaries were removed after day 4 and day 7 in culture and processed for histological analysis. The number of primordial and growing follicles was counted. Primordial follicle count in the presence of AMH and cisplatin is higher than in the presence of cisplatin alone (results not shown).

Thus, treatment with AMH is suitable for fertile women undergoing chemotherapy as it offers an advantageous platform for preserving their fertility and prolong maintenance of the ovarian function due to larger follicle stockpile which survive treatment.

It is worth noting that in order to obtain valuable data, studies related to primordial follicles should be conducted in primordial follicles from ovaries that completed their perinatal packaging, namely, ovaries from mice that are at least 10 days old since normal ovarian follicle dynamics only begin after about 10 days. At that stage the balanced dynamic of follicle activation/suppression is stabilized. Ovaries at an earlier stage (such as ovaries of 2-day-old mice) have not completed their perinatal packaging, nor reached normal ovarian follicle dynamics, leaving many ‘naked oocytes’.

### Example 2: AMH Improves Growing:Dormant Follicles Ratio Under Chemotherapy

The ratio of growing/dormant follicles was examined in ovaries treated with the chemotherapy drug with and without AMH. Significant differences between the treatments were observed on day 4 and 7. The ratio of growing to dormant follicles was greatest in the ovaries exposed to PM alone. This ratio was significantly improved in ovaries exposed to PM+AMH (p<0.05).

Overall, the results indicate that AMH reduces chemo-induced follicle activation, suggesting its potential in protecting follicle reserve in young female cancer patients.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-18. (canceled)

19. A method of inhibiting premature follicle activation comprising administering to a subject in need thereof a therapeutically effective amount of a pharmaceutical composition comprising a compound selected from the group consisting of anti-mullerian hormone, anti-mullerian hormone agonist, and antiMIR of anti-mullerian hormone, wherein the premature follicle activation is induced by an acute insult.

20. The method of claim 19, wherein the compound is anti-mullerian hormone.

21. The method of claim 19, wherein said acute insult is selected from the group consisting of treatment, agent, disease or a combination thereof.

22. The method of claim 21, wherein said treatment is transplantation of ovarian tissue or whole ovary.

23. The method of claim 22, further comprising transplanting ovarian tissue or whole ovary.

24. The method of claim 23, wherein said pharmaceutical composition is administered prior to, or during, said transplanting.

25. The method of claim 21, wherein said agent is a follicle loss inducing agent.

26. The method of claim 25, wherein said agent is a chemotherapeutic drug.

27. The method of claim 19, wherein said acute insult comprises chemotherapy, radiotherapy or a combination thereof.

28. The method of claim 21, wherein said disease is an accelerated follicle activation disorder.

29. The method of claim 28, wherein the disease is selected from endometriosis, galactosemia, Turner syndrome and an autoimmune disease.

30. The method of claim 19, further comprising administering to said subject at least one follicle reserve protective compound.

31. The method of claim 19, wherein said subject is a female subject in her reproductive years.

32. A kit for inhibiting premature follicle activation induced by an acute insult in a subject in need thereof comprising:
(i) a first packaging containing a pharmaceutical composition comprising a compound selected from the group consisting of anti-mullerian hormone, anti-mullerian hormone agonist, and antiMIR of anti-mullerian hormone; and
(ii) written instructions of use of said pharmaceutical composition for inhibiting in said subject premature follicle activation induced by a medical treatment or a disease.

33. The kit of claim 32, further comprising a second packaging containing a pharmaceutical composition comprising a follicle loss inducing agent.

34. The kit of claim 32, further comprising a third packaging containing at least one follicle reserve protective compound.

35. The kit of claim 32, wherein said follicle loss inducing agent is a chemotherapeutic agent.

36. The kit of claim 32, wherein the compound is anti-mullerian hormone.

37. The kit of claim 32, wherein said medical treatment is transplantation of ovarian tissue or whole ovary.

38. The kit of claim 32, wherein said disease is an accelerated follicle activation disorder.
